(19) United States
(12) Patent Application Publication
(10) Pub. No.: US 2022/0074918 A1
(43) Pub. Date: Mar. 10, 2022

(54) Toilet Tracking Timing of Excreta Events

(71) Applicant: Medic, Inc., Provo, UT (US)

(72) Inventors: David R. Hall, Provo, UT (US); K. Jeffrey Campbell, Spanish Fork, UT (US)

(73) Assignee: Medic, Inc., Provo, UT (US)

(21) Appl. No.: 17/468,619

(22) Filed: Sep. 7, 2021

Related U.S. Application Data
(60) Provisional application No. 63/074,643, filed on Sep. 4, 2020.
(60) Provisional application No. 63/074,608, filed on Sep. 4, 2020.

(51) Int. Cl. G01N 33/483; G01N 33/493; A61B 5/20; A61B 10/00

(52) U.S. Cl. CPC G01N 33/4833; G01N 33/493; E03D 9/00; A61B 10/0038; A61B 5/208

(57) ABSTRACT

A system and method for assessing toilet user health and wellness is disclosed. The system comprises a toilet bowl adapted to receive excreta from a user; a sensor which detects at least one property of the user or excreta and records a timestamp associated with the property; and a processor which analyzes the property and uses the time stamp to correlate the property with data from previous events to assess one or more health or wellness conditions; wherein the correlation of the date stamp comprises correlating events that take place during the same segment of the day. The method comprises providing a system comprising a toilet bowl adapted to receive excreta from a user; a sensor which detects at least one property of the user or excreta and records the time of the excreta event; and a processor which analyzes the timing of excreta events and correlates the receiving excreta into the toilet bowl; obtaining data from the sensor and recording it; correlating data from separate events based on the time of day when the events occurred; and using the correlated data to monitor trends and analyze trends to assess the user's health and wellness.

### CROSS-REFERENCE TO RELATED APPLICATIONS

This application claims the benefit under 35 U.S.C. 119(e) of U.S. Provisional Patent Application Nos. 63/074,643 titled “Toilet Tracking of Event Timing” filed on 4 Sep. 2020, and 63/074,608 titled “Toilet Wastewater Collection and Testing” filed on 4 September 2020, which disclosures are incorporated herein by reference in their entireties.

### TECHNICAL FIELD

The present disclosure relates to analytical toilets. More particularly, it relates to analytical toilets equipped to provide health and wellness information to the user.

### BACKGROUND

The ability to track an individual's health and wellness is currently limited due to the lack of available data related to personal health. Many diagnostic tools are based on examination and testing of excreta, but the high cost of frequent doctor's visits and/or scans make these options available only on a very limited and infrequent basis. Thus, they are not widely available to people interested in tracking their own personal wellbeing.

Toilets present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

Just a few examples of smart toilets and other bathroom devices can be seen in the following U.S. Patents and Published Applications: U.S. Pat. No 9,867,513, entitled “Medical Toilet With User Authentication”; U.S. Pat. No. 10,123,784, entitled “In Situ Specimen Collection Receptacle In A Toilet And Being In Communication With A Spectral Analyzer”; U.S. Pat. No. 10,273,674, entitled “Toilet Bowl For Separating Fecal Matter And Urine For Collection And Analysis”; US 2016/0000378, entitled “Human Health Property Monitoring System”; US 2018/0020984, entitled “Method Of Monitoring Health While Using A Toilet”; US 2018/0055488, entitled “Toilet Volatile Organic Compound Analysis System For Urine”; US 2018/0078191, entitled “Medical Toilet For Collecting And Analyzing Multiple Metrics”; US 2018/0140284, entitled “Medical Toilet With User Customized Health Metric Validation System”; and US 2018/0165417, entitled “Bathroom Telemedicine Station.” The disclosures of all these patents and applications are incorporated by reference in their entireties.

### SUMMARY

In a first aspect, the disclosure provides a system for assessing toilet user health and wellness. The system comprises a toilet bowl adapted to receive excreta from a user; a sensor which detects at least one property of the user or excreta and records a timestamp associated with the property; and a processor which analyzes the property and uses the time stamp to correlate the property with data from previous events to assess one or more health or wellness conditions; wherein the correlation of the date stamp comprises correlating events that take place during the same segment of the day.

In a second aspect, the disclosure provides a method of using the same. The method comprises providing a system comprising a toilet bowl adapted to receive excreta from a user; a sensor which detects at least one property of the user or excreta and records the time of the excreta event; and a processor which analyzes the timing of excreta events and correlates the receiving excreta into the toilet bowl; obtaining data from the sensor and recording it; correlating data from separate events based on the time of day when the events occurred; and using the correlated data to monitor trends and analyze trends to assess the user's health and wellness.

Further aspects and embodiments are provided in the foregoing drawings, detailed description, and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is an isometric view of one exemplary embodiment of a toilet.

FIG. 2 is a top view of the toilet of FIG. 1.

FIG. 3 is a view of the bottom of the seat and lid of the toilet of FIG. 1.

FIG. 4 is a view from the side of the toilet of FIG. 1 with the cover removed.

FIG. 5 is an isometric view of a second exemplary embodiment of a toilet.

FIG. 6 is a top view of the toilet of FIG. 7.

FIG. 7 is a view of the bottom of the seat of the toilet of FIG. 7.

FIG. 8 is a partial view of the toilet of FIG. 7 with the cover removed.

FIG. 9 is a detail view of one exemplary embodiment of a handle.

FIG. 10 is a plot of example general event timing information.

FIG. 11 is a plot of the example general event timing information from FIG. 10 in relation to an event source.

FIG. 12 is a plot of example excreta event timing information.

FIG. 13 is a plot of the example excreta event timing information from FIG. 12 and excreta classification.

FIG. 14 is a set of sample plots of excreta volume and flow rate data.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “toilet” is meant to refer to any device or system for receiving human excreta, including urinals.

As used herein, the term “bowl” refers to the portion of a toilet that is designed to receive excreta.

As used herein, the term “base” or “frame” refers to the portion of the toilet below and around the bowl supporting it.

As used herein, the term “user” refers to any individual who interacts with the toilet and deposits excreta therein.

As used herein, the term “excreta” refers to any substance released from the body of a user including urine, feces, menstrual discharge, saliva, expectorate, and anything contained or excreted therewith.

As used herein, the term “excretion profile” is meant to refer collectively to the rate of excretion at any moment in time of an excretion event and the total volume or mass of excreta as a function of time during an excretion event. The terms “defecation profile” and “urination profile” refer more specifically to the separate measurement of excreta from the anus and urethra, respectively.

As used herein, the term “sensor” is meant to refer to any device for detecting and/or measuring a property of a person or of a substance regardless of how that property is detected or measured, including the absence of a target molecule or characteristic. Sensors may use a variety of technologies including, but not limited to, MOS (metal oxide semiconductor), CMOS (complementary metal oxide semiconductor), CCD (charge-coupled device), FET (field-effect transistors), nano-FET, MOSFET (metal oxide semiconductor field-effect transistors), spectrometers, volume measurement devices, weight sensors, temperature gauges, chromatographs, mass spectrometers, IR (infrared) detector, near IR detector, visible light detectors, and electrodes, microphones, load cells, pressure gauges, PPG (photoplethysmogram), thermometers (including IR and thermocouples), rheometers, durometers, pH detectors, scent detectors gas, and analyzers.

As used herein, the term “imaging sensor” is meant to refer to any device for detecting and/or measuring a property of a person or of a substance that relies on electromagnetic radiation of any wavelength (e.g., visible light, infrared light, x-ray) or sound waves (e.g., ultrasound) to view the surface or interior of a user or substance. The term “imaging sensor” does not require that an image or picture is created or stored even if the sensor is capable of creating an image.

As used herein, the term “data connection” and similar terms are meant to refer to any wired or wireless means of transmitting analog or digital data and a data connection may refer to a connection within a toilet system or with devices outside the toilet.

As used herein, the terms “biomarker” and “biological marker” are meant to refer to a measurable indicator of some biological state or condition, such as a normal biological processes, pathogenic processes, or pharmacologic responses to a therapeutic intervention. Some biomarkers are related to individual states or conditions. Other biomarkers are related to groups or classifications or states or conditions. For example, a biomarker may be symptomatic of a single disease or of a group of similar diseases that create the same biomarker.

As used herein, the term “analyte” is meant to refer to a substance whose chemical constituents are being identified and measured.

As used herein, a “fluidic circuit” is meant to refer to the purposeful control of the flow of a fluid. Often, this is accomplished through physical structures that direct the fluid flow. Sometimes, a fluidic circuit does not include moving parts.

As used herein, a “fluidic chip” is meant to refer to a physical device that houses a fluidic circuit. Often, a fluidic chip facilitates the fluid connection of the fluidic circuit to a body of fluid.

As used herein, the term “microfluidics” is meant to refer to the manipulation of fluids that are contained to small scale, typically sub-millimeter channels. The prefix “micro” used with this term and others in describing this invention is not intended to set a maximum or a minimum size for the channels or volumes.

As used herein, the prefix “nano” is meant to refer to something in size such that units are often converted to the nano-scale for ease before a value is provided. For example, the dimensions of a molecule may be given in nanometers rather than in meters.

As used herein, “bind” and similar variants are meant to refer to the property of facilitating molecular interaction with a molecule, such as interaction with a molecular biomarker.

As used herein, “event” is meant to refer to a detectable phenomenon, especially an input to or output from the system or an output from a user of the system. Example events include a user depositing excreta into a toilet, other discrete portions of biological processes, and discrete portions of sensor measurements by the system of the user or environment. Various sets of rules may be used to define an event and use of one set of rules to define an event does not necessarily exclude it from being defined in an additional way by a different set of rules.

As used herein, “timestamp” and similar terms are meant to refer to the use of a time reference to distinguish when an event occurred. This reference may be absolute (e.g., a clock, calendar, or method of time keeping outside the system) and/or relative (e.g., time lapsed using a timer or by comparison to another event within the system). A timing determination may be done in terms of a moment and/or a period of time. The timestamp may be anywhere from the beginning to the end of the event. A time reference may include divisions of various length, such as parts of seconds, seconds, minutes, hours, days, months, and years. “Time” may be used to refer to any of these divisions. When used together, “time” and “date” can be considered as follows: “time” refers to the time of day when an events occurs (e.g., hour, minute, etc.) and “date” refers to the calendar date, possibly including day of the week as well. The timestamp of an event may be classified based on time of day when it occurs (e.g., morning, afternoon, evening, nighttime, etc.), the individual user's schedule (e.g., sleep patterns, work schedule, etc.), or location (e.g., home, work, gym, store, etc.).

### Exemplary Embodiments

The present disclosure relates to tracking the timing of biological process, such as excreta events in a toilet, and using the timing as part of the analysis of toilet user data. It includes doing so over the course of multiple uses of the toilet. It also includes doing so within a single use of the toilet. The system may track when excreta events happen and uses that to analyze data, especially with frequency of the events throughout a day. Frequency can be analyzed to help identify potential conditions, including prostate issues, diabetes, anxiety, and other health and wellness states that manifest by or related to how often someone deposits excreta and volume deposited.

In a preferred embodiment, excreta events on separate days are correlated based on the time of day they occur (e.g., upon waking, afternoon or evening, during normal sleep hours) based on individual schedules to establish baseline measurements, trends, and deviations from the normal baseline pattern. The individual's schedule may be based on information provided by the user and/or determined based on measured patterns. Other devices, such as sleep monitors or CPAP machines, may be used to determine sleep patterns and classify toilet uses that interrupt sleep. A user's GPS capable device (e.g., smart phone) may also be used to provide data about location (e.g., home, work, and other locations) to classify excreta events. Phone apps that record physical activity (e.g., pedometers) or that are used to log diet and/or exercise, medical databases (e.g., from the user's health care providers), smart medical devices (e.g., blood pressure meters), smart exercise machines, and other sources of information may be used to obtain data about the user and correlate it with data from the toilet. The data from events across multiple days to be compiled based on this classification and analyzed separately from data in other classifications to provide greater insight. For example, excreta events that interrupt sleep may be analyzed separately. Data from events during the afternoon or evening hours, for example, can be analyzed without being skewed by data from the first event after awaking or vice versa.

One benefit of the present disclosure is using the data from multiple uses of the toilet to help assess a user's health and wellness as well as analyzing the data by considering what part of the user's daily routine the data is gathered. For example, there are health and wellness conditions which correlate with certain urination or defecation frequencies and/or volume during a period of time longer than a single use of the toilet, including urinary tract infections, diabetes, prostate issues, inflammatory bowel disease, age, pregnancy, dietary changes, and hydration level (see https://www.mayoclinic.org/symptoms/frequent-urination/basics/causes/sym-20050712 for more information on urination and https://www.healthline.com/health/how-many-times-should-you-poop-a-day#factors for more information on defecation). Additionally, excreta is often different during different parts of the day. For example, the first urination after waking from a long sleep is generally more concentrated. Furthermore, the time synchronous data can be used to create trend data for the user and then used as a reference to recognize changes from the trend. Additionally, the user data recorded by the system can be additionally informed by user input, such as if the user used a toilet which doesn't report user data to the system. As another example, the system may be able to recognize whether the user has regular physical activity.

Another benefit of the present disclosure is using the timeline of data from a single use of the toilet to help analyze the data. For example, when a user first approaches the toilet, they may have undergone some exertion to get to the toilet. Similarly, depositing excreta into the toilet may have some exertion associated with that act. In both examples, the exertion can be used to explain elements of the data, including higher temperature, blood pressure, and/or heart rate as well as changes to such properties. In another example, sensors in the system may measure properties of the user that can be associated with a change to the user's conditions, such as a leg falling asleep. Such data could be more informative if compared to other data, such as the timing of a change in the user's position or posture.

In one preferred embodiment, the system tracks when excreta leaves a user and/or enters the toilet. The data can be used in combination with other data about the excreta or user to create trend information, including excreta frequency and total volume. This data could also be augmented through external input, such as allowing the user to let the system know they used a toilet not integrated with the system.

In one embodiment, the timing of an excreta deposit is synced with when other data is captured about the user. The toilet may include sensors for detecting physical properties of the user, such as heart rate, blood pressure, impedance, tremors, electromagnetic imaging, and sounds and vibrations coming from the user. The toilet may also detect properties of excreta itself, such as color, temperature, density, various biomarkers, and various analytes.

In a preferred embodiment, the system additionally classifies excreta by type and uses that as part of the data analysis.

In some preferred embodiments, the system includes a processor to receive, process, and/or analyze data about the user, including data about excreta. This may be a single unit or multiple units which work to accomplish these goals. In one preferred embodiment, there is a processor in the toilet. In another preferred embodiment, data is sent to a remote processor, such as in a cloud-based system. A processor may be in a mobile device such as a cell phone or tablet. Another embodiment may use a combination of these and/or other processors. In one preferred embodiment, the processor is part of a neural network which receives data and outputs results it may correlate with the user.

The timing of excreta events verses when other measurements are taken can be relevant for many reasons. For example, targeted biomarkers in a sample from the user may increase or decrease over time. As another example, cardiovascular and/or other body-state measurements may be affected by strain associated with expelling excreta from the body. Syncing the timing of any of these measurements with the timing of excreta events or other physical exertions can improve the information value of the measurement, such as by calibrating the measurement.

In one preferred embodiment, the timing data is in the form of a date and time. For example, there are many common data and time formats based on UTC (Coordinated Universal Time). UTC-based formats can include the following representations:

YYYY=four-digit year

MM=two-digit month (01=January, etc.)

DD=two-digit day of month (01 through 31)

hh=two digits of hour (00 through 23) (am/pm NOT allowed)

mm=two digits of minute (00 through 59)

ss=two digits of second (00 through 59)

s=one or more digits representing a decimal fraction of a second

TZD=time zone designator (Z or +hh:mm or −hh:mm)

One example format that uses these representations is as follows: YYYY-MM-DDThh:mm:ss.s TZD and an example in this format is 1997-07-16T19:20:30.45+01:00. This example represents 7:20 pm and 30 45/100 seconds on Jul. 16, 1997 in the time zone 1 hour ahead of UTC. Non-UTC date and time formats can include similar components. The selection of the format includes considerations outside the scope of invention for this disclosure.

The following US Patent and Provisional Patent Applications discuss various implementations of sensors which may be able to detect and/or measure properties of a user in various embodiments of toilets: U.S. patent application Ser. 16/818,900 titled “Toilet with Vascular Health Reporting” filed 13 Mar. 2020; U.S. patent application Ser. 16/888,024 titled “Toilet Configured to Distinguish Excreta Type” filed 29 May 2020; Provisional Application No. 62/979,803 titled “Analytical Toilet for Assessing Analytes in Excreta” filed 21 Feb. 2020; Provisional Application No. 62/993,648 titled “Analytical Toilet for Detecting Viruses in Feces” filed 23 Mar. 2020; Provisional Application No. 63/002,200 titled “Analytical Toilet for Detecting Viruses in Urine” filed 30 Mar. 2020; Provisional Application No. 63/055,595 titled “Customized Survey for User of Analytical Toilet” filed 23 Jul. 2020. Each of these applications are incorporated into the specification herein by reference in their entirety.

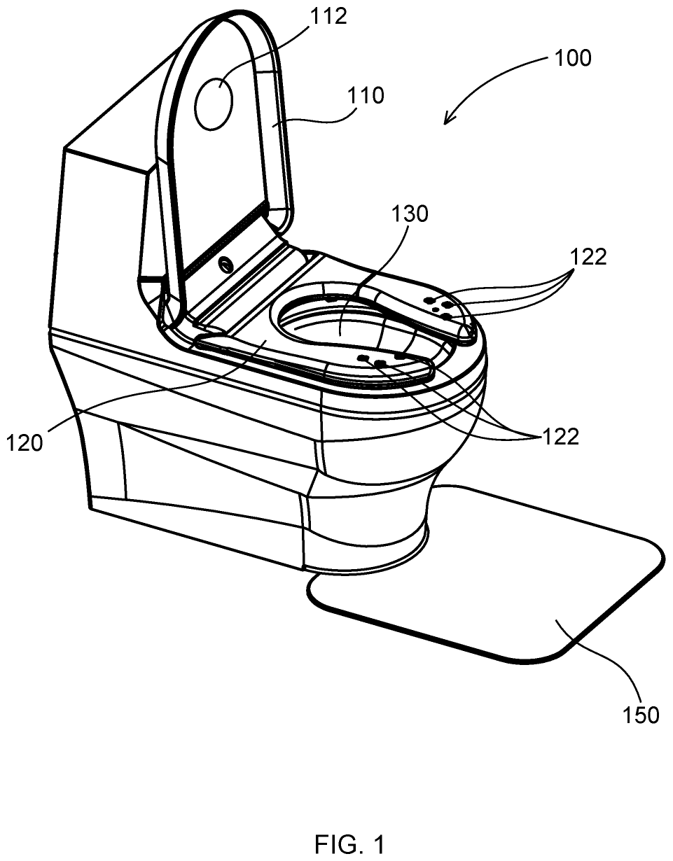

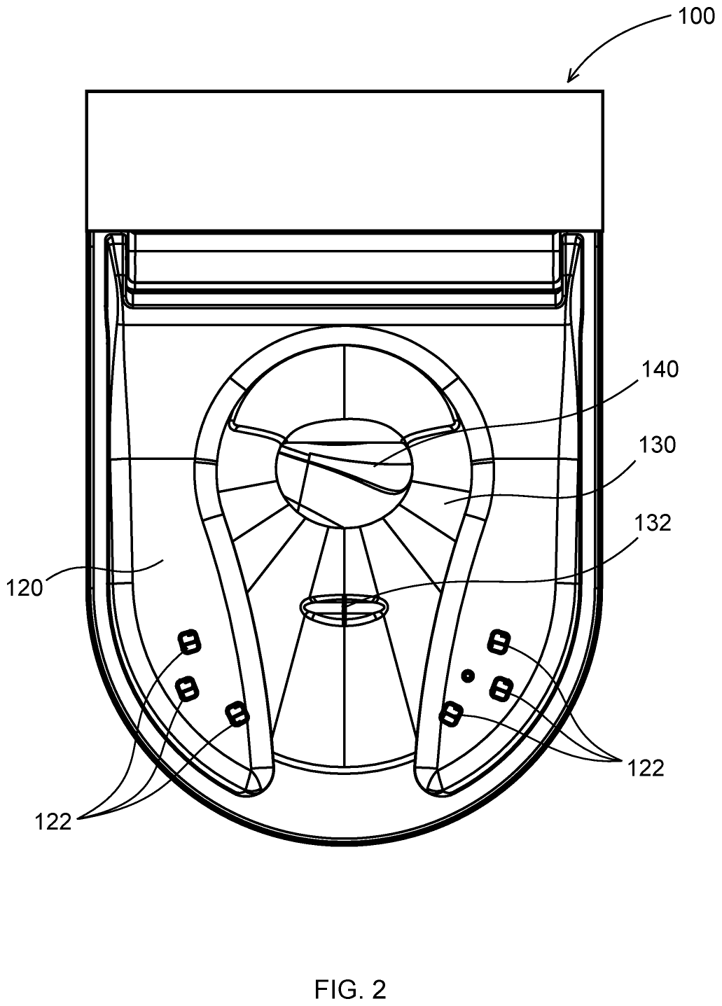

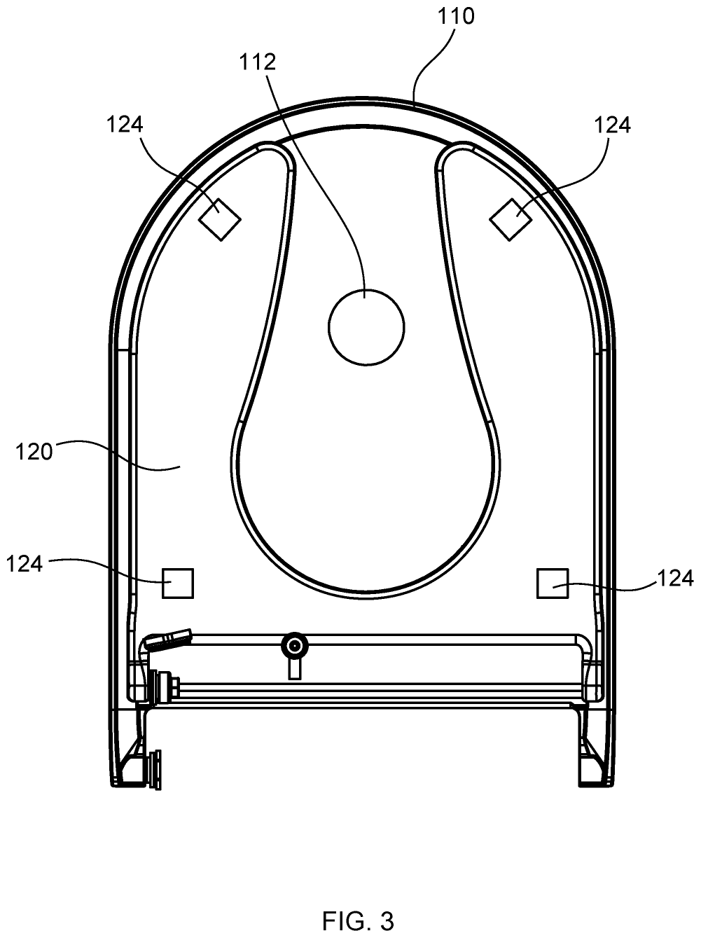

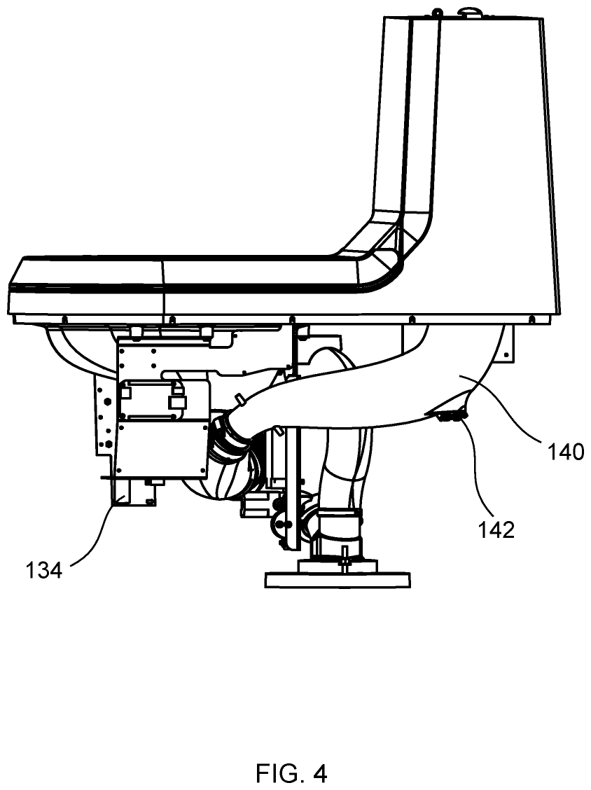

Now referring to FIGS. 1-4, one preferred embodiment of the toilet used in the system is shown. FIG. 1 shows an isometric view of toilet 100 with lid 110 open, showing seat 120 with multiple PPG sensors 122, bowl 130, and foot scale 150. Foot scale 150 may have a variety of sensors, such as sensors to determine a user's weight, image sensors, and electrical contacts. FIG. 2 shows a top view of toilet 100 with lid 110 open, showing seat 120 with multiple PPG sensors 122, bowl 130, and excreta volume measure tube 140. Bowl 130 includes urine slit 132, which captures urine for readings by spectrometer 134 and may also capture a sample of urine and remove the sample from the bowl to another location. FIG. 3 is a detail view of the underside of seat 120 with lid 110 behind seat 120. On the underside of seat 120 are weight sensors 124. Shown on lid 110 is acoustic sensor 112, which includes a microphone for recording audio sounds from a user's trunk portion of the body; acoustic sensor 112 could be supplemented with another type of sensor. FIG. 4 is a detail view showing some of the internal components of toilet 100, including excreta volume measure tube 140, excreta tube volume sensor 142, and spectrometer 134. The toilet may also have other sensors to detect properties of a user, including from a user's excreta. The toilet may also have a way of sampling and testing the wastewater in the bottom of bowl 130 and excreta measure tube 140 before and/or after it the water has interacted with excreta.

In one preferred embodiment, a user walks onto scale 150, and sits down on seat 120, leaving their feet on scale 150. While the user is using the toilet, PPG sensors 122 monitor the user's upper legs,; weight sensors 124 monitor the portion of the user's weight on seat 120 (including minor, apparent fluctuations that are a result of a user's cardiovascular activity), weight sensors 154 monitor the portion of the user's on foot scale 150, and bioimpedance sensors 152 determine the user's bioimpedance. Timestamped data from one or more sensors can be used as part of the data analysis to assess the user's health and wellness.

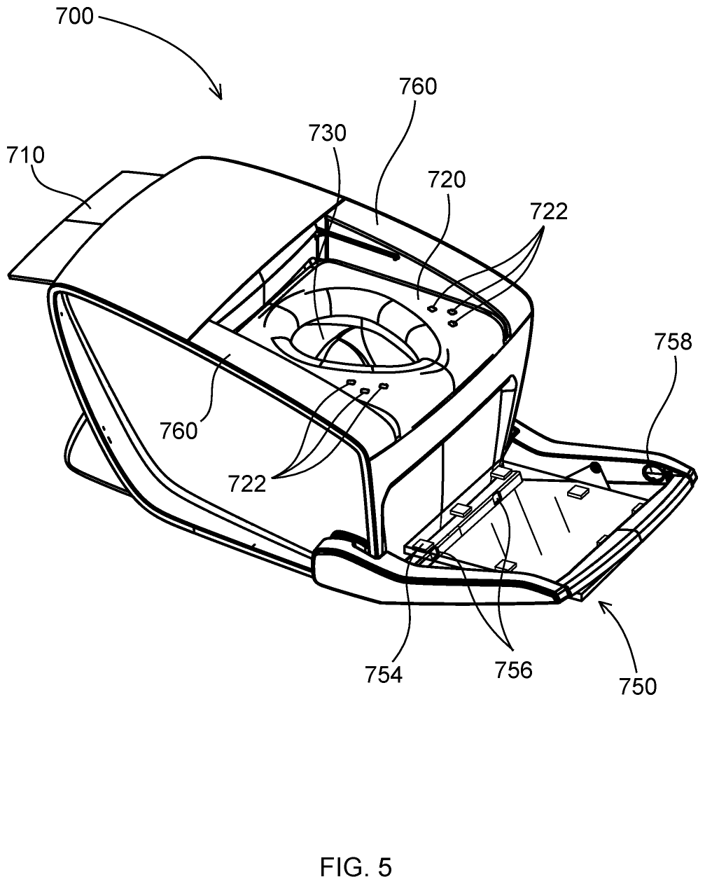

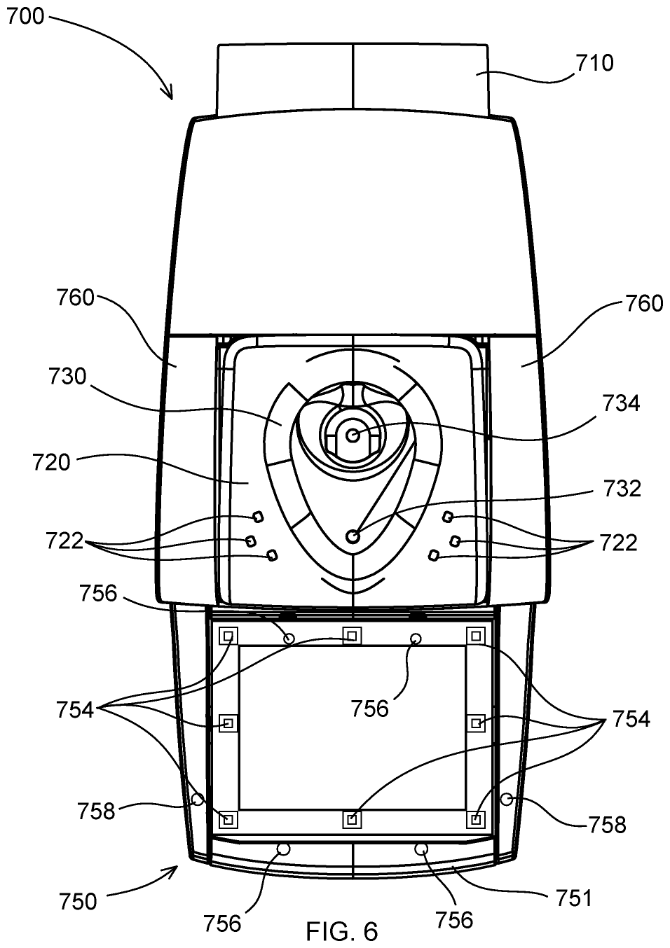

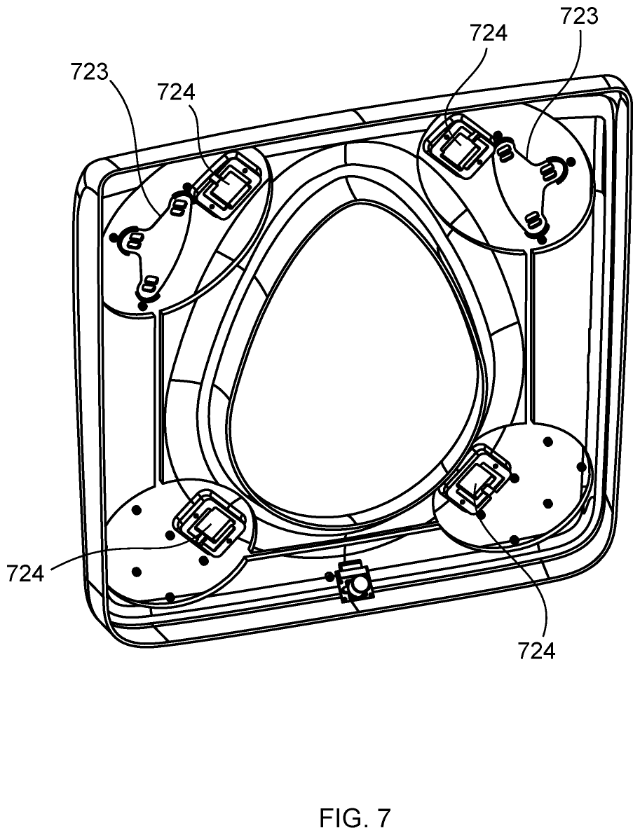

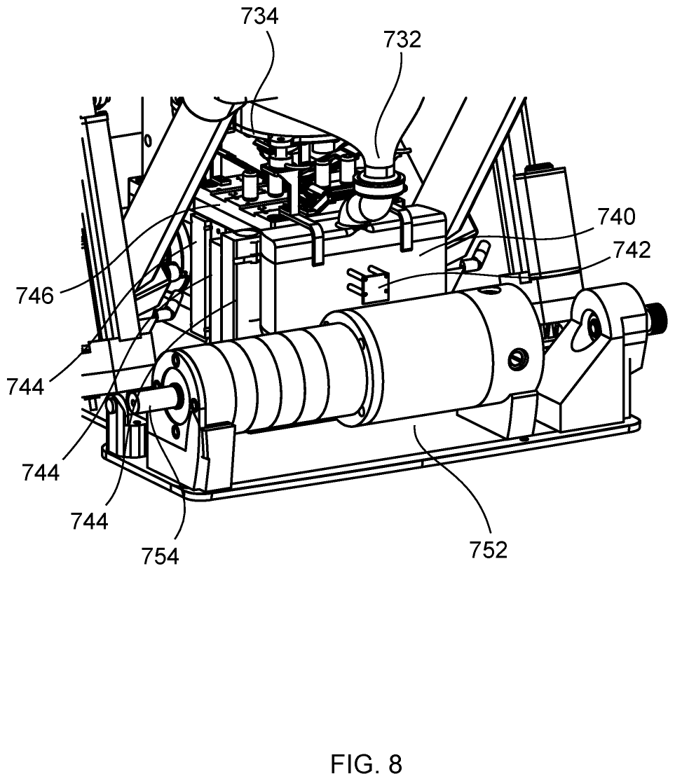

FIGS. 5-8 show another embodiment of the toilet. FIG. 5 shows an isometric view of toilet 700 with lid 710 open, showing seat 720 with multiple PPG sensors 722, bowl 730, foot platform 750, and handles 760. FIG. 6 shows a top view of toilet 700 with lid 710 open, showing seat 720 with multiple PPG sensors 722, bowl 730, foot platform 750, and handles 760. Bowl 730 includes urine receptacle 732 and fecal depository 734. In one preferred embodiment, handles 760 are in a recessed position and can be raised up relative to the toilet. FIG. 7 is a detail view of the underside of seat 720 showing weight sensors 724. FIG. 8 is a detail view showing some of the internal components of toilet 700, including urine receptacle 732, fecal depository 734, urine chamber 740, urine spectrometer 742, science centers 744, fluid chip receptacle 746, foot platform motor and sensor 752, foot platform motor shaft 753. Foot platform 750 includes frame 751, a glass plate resting on multiple weight sensors 754, foot IR sensors 756, and foot near-IR sensors 758. Additionally, visible light sensors may also detect and the feet to obtain data on them. In one preferred embodiment, science centers 744 and fluid chip receptacle 746 are used as part of excreta analysis in toilet 700, including urine samples and emulsified or otherwise processed excreta.

In one preferred embodiment, a user walks onto platform 750, sits down on seat 720, and platform 750 raises up so the user's feet easily stay on the glass plate. While the user is using the toilet, PPG sensors 722 monitor the user's upper legs; weight sensors 724 monitor the portion of the user's weight on seat 720-including minor, apparent fluctuations that are a result of a user's cardiovascular activity or other body tremors; weight sensors 754 monitor the portion of the user's on foot platform 750, and sensors 756 and 758 monitor the user's feet and lower legs. After excreta is deposited into the toilet, urine flows through urine receptacle 732 into urine chamber 740 where the urine can be sampled and/or tested; there may or may not be wastewater in urine chamber 740 with the urine. Feces is received by fecal depository 734 where it can be sampled and/or tested; there may or may not be wastewater on fecal depository 734 with the feces. At some point after, flush water can act to carry feces off fecal depository 734 to a secondary processing area where the feces and/or waste water can be sampled and/or tested; the water for the wastewater may solely be from the flush water or there may have been water already in the secondary processing area. Additionally, water jets and/or other process can be used to comminute the feces on the fecal depository or in the secondary processing area. Alternatively, the flush water and feces may go directly from the depository out of the toilet. There may also be toilet paper and other non-fecal/non-urine waste with the urine and/or feces. Timestamped data from one or more sensor can be used as part of the data analysis to assess the user's health and wellness.

In one preferred embodiment, sensors 756 and 758 are able to detect properties of the foot, including foot size and shape, coloring, and subdermal vascular properties. These sensors could be image sensors that capture visible light, IR light, or another part of the electromagnetic spectrum. They may also emit visible light, IR light, or another part of the electromagnetic spectrum. Images created from these sensors can undergo image recognition analysis, the results of which can be compared to preexisting data on the same to generate a report on a user's health. Preferably, the report includes information relative to a user's vascular health. Preferably, the comparison is performed by a neural net which has been trained to recognize commonalities to and differences from preexisting images. When the preexisting images are coupled with known health states and/or conditions of the person from whom the images came, the neural net can suggest correlations between the user's images and health states and/or conditions (including neutral or positive ones). Additionally, when the neural net has examined previous data from the same user, the neural net can compare the user's prior state to his or her current state to report on the relative change. Therefore, it may be useful for user data to be averaged, have the mean taken, used in creating trend data, or otherwise be used in creating a baseline against which to compare new user data as it is generated.

FIG. 9 shows an embodiment of a handle that could accompany a toilet. Handle 1160 includes electrical lead 1162 and PPG sensor 1164. Electrical lead 1162 could be a lead for a bioimpedance sensor and/or an ECG (aka EKG) sensor. In one preferred embodiment, a handle would be connected to a cord (with wiring) that connects to the toilet. Alternatively, a handle could be wireless and in digital communication with a controller that is also in digital communication with other of the toilet sensors. And another preferred embodiment a handle would be mounted to a structure adjacent to the toilet bowl. In either embodiment a second handle they also do used. A second handle may originate from the same connection point to the toilet or a location symmetrically opposite or mirrored from the first handle.

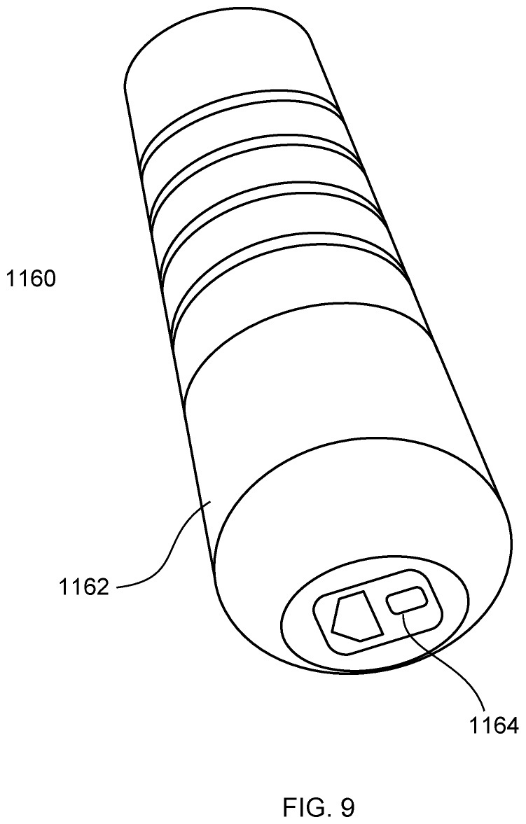

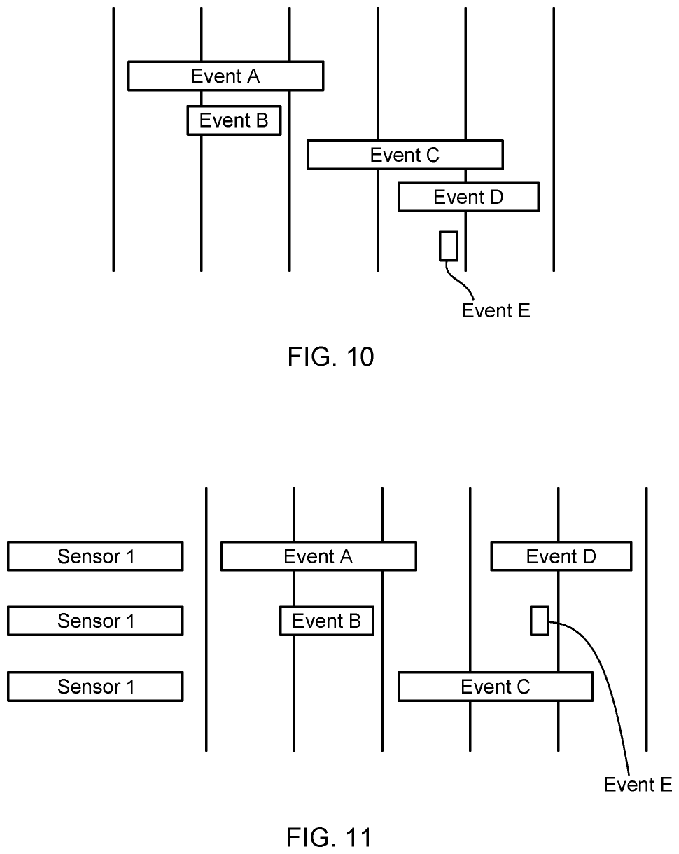

FIGS. 10-13 show various examples of events of various duration that happen with their chronological relationship to each other. The chronological reference may include an event or a clock. In FIG. 10, general events A-E are plotted relative to the same chronological reference, allowing each event to be chronologically compared to the others to determine relative characteristic such as start times, durations, end times, and overlap between events. In FIG. 11, the events from FIG. 10 are associated with a sensor which measured or recorded the event.

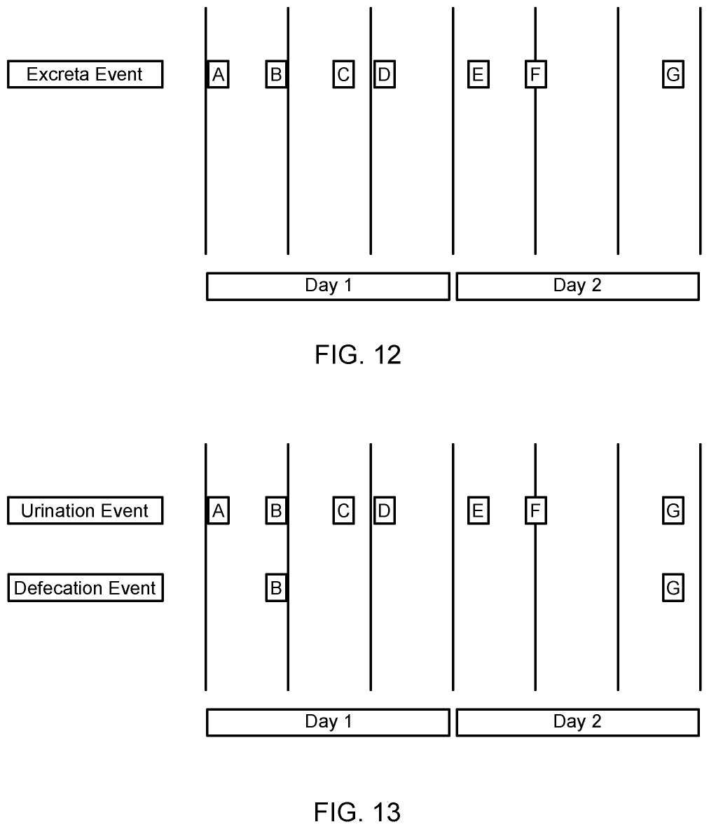

In FIG. 12, a set of excreta events A-G are plotted over a 2-day period. With this plot, frequency and other timings can be established. Additionally, each excreta event may have additional data, such as excreta event duration, excreta volume, a determination of how much strain the user may have been under, and results from excreta analysis (including biomarker and analyte measurements). In FIG. 13, excreta events A-G are classified as Urination and/or Defecation Events.

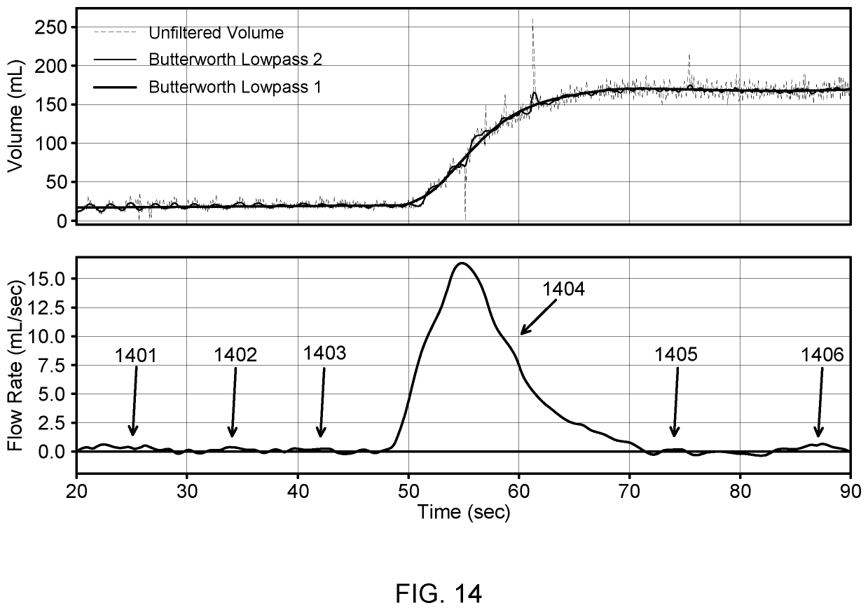

In FIG. 14, sensor data from one use of a toilet embodiment is used to create a time-based plot of the flow rate and total volume of excreta deposited for the use session. The various plots on the volume graph represent different types of data processing. The plot of the flow rate shows a flow rate for the excreta deposited extrapolated from the sensor data and data processing. Items 1401-1406 are potential excreta event candidates. Additional information, including identifying and classifying of excreta event, may be found in the previously referenced U.S. patent application Ser. No. 16/888,024.

All patents, published patent applications, and other publications referred to herein are incorporated herein by reference. The invention has been described with reference to various specific and preferred embodiments and techniques. Nevertheless, it is understood that many variations and modifications may be made while remaining within the spirit and scope of the invention.

What is claimed is:

1. A system for assessing toilet user health and wellness comprising:
a toilet bowl adapted to receive excreta from a user;
a sensor which detects at least one property of the user or excreta and records a timestamp associated with the property; and
a processor which analyzes the property and uses the time stamp to correlate the property with data from previous events to assess one or more health or wellness conditions;
wherein the correlation of the date stamp comprises correlating events that take place during the same segment of the day.

2. The system of claim 1, wherein the sensor comprises at least one of CCD, MOS, CMOS, spectrometers, chromatographs, FET, nanoFET, MOSFET, mass spectrometers, electrodes, microphones, load cells, pressure gauges, PPG, thermometers, rheometers, durometers, pH detectors, scent detectors, imaging cameras, spectrometers, volume measurement devices, weight sensors, temperature gauges, chromatographs, and gas analyzers.

3. The system of claim 1, wherein the sensor measures the volume of urine received by the bowl and wherein the correlation to the condition is based on the volumes a user urinates over the course of one or more urination events.

4. The system of claim 1, wherein the sensor measures the volume of feces received by the bowl and wherein the correlation to the condition is based on the volumes a user defecates over the course of one or more defecation events.

5. The system of claim 1, wherein the property detected is one or more of excreta content, excreta weight, excreta volume, excreta temperature, excreta density, and excreta flow rate.

6. The system of claim 1, wherein the condition comprises one or more of hydration, diabetes, pregnancy, prostate health, kidney health, urinary tract infection, inflammatory bowel disease, diarrhea, and constipation.

7. The system of claim 1, wherein the correlation of the date stamp comprises correlating events on separate days that take place during the same segment of the day.

8. The system of claim 1, wherein the correlation to the condition is based on the frequency which a user urinates over the course of one or more days.

9. The system of claim 1, wherein the correlation to the condition is based on the frequency which a user defecates over the course of one or more days.

10. The system of claim 1, wherein the excreta is analyzed a period of time after its receipt into the toilet bowl and the period of time is used to calibrate the results of the analysis.

11. A method of assessing the health and wellness of a toilet user comprising:
providing a system comprising:
a toilet bowl adapted to receive excreta from a user;
a sensor which detects at least one property of the user or excreta and records the time of the excreta event; and
a processor which analyzes the timing of excreta events and correlates the receiving excreta into the toilet bowl;
obtaining data from the sensor and recording it;
correlating data from separate events based on the time of day when the events occurred; and
using the correlated data to monitor trends and analyze trends to assess the user's health and wellness.

12. The method of claim 11, wherein the sensor comprises at least one of CCD, MOS, CMOS, spectrometers, chromatographs, FET, nanoFET, MOSFET, mass spectrometers, electrodes, microphones, load cells, pressure gauges, PPG, thermometers, rheometers, durometers, pH detectors, scent detectors, imaging cameras, spectrometers, volume measurement devices, weight sensors, temperature gauges, chromatographs, and gas analyzers.

13. The method of claim 11, wherein the sensor measures the volume of urine received by the bowl and wherein the correlation to the condition is based on the volumes a user urinates over the course of one or more urination events.

14. The method of claim 11, wherein the sensor measures the volume of feces received by the bowl and wherein the correlation to the condition is based on the volumes a user defecates over the course of one or more defecation events.

15. The method of claim 11, wherein the property detected is one or more of excreta content, excreta weight, excreta volume, excreta temperature, excreta density, and excreta flow rate.

16. The method of claim 11, wherein the condition comprises one or more of hydration, diabetes, pregnancy, prostate health, kidney health, urinary tract infection, inflammatory bowel disease, diarrhea, and constipation.

17. The method of claim 11, wherein the correlation of the date stamp comprises correlating events on separate days that take place during the same segment of the day.

18. The method of claim 11, wherein the correlation to the condition is based on the frequency which a user urinates over the course of one or more days.

19. The method of claim 11, wherein the correlation to the condition is based on the frequency which a user defecates over the course of one or more days.

20. The method of claim 11, wherein the excreta is analyzed a period of time after its receipt into the toilet bowl and the period of time is used to calibrate the results of the analysis.
